(19) United States
(12) Patent Application Publication
(10) Pub. No.: US 2005/0240114 A1
(43) Pub. Date: Oct. 27, 2005

(54) Method and system for achieving heart rate variability coherence during exercise

(76) Inventor: Stephen Bennett Elliott, Allen, TX (US)

Correspondence Address: Stephen Bennett Elliott, 702 Buffalo Springs Drive, Allen, TX, 75013, US

(21) Appl. No.: 10/829,079

(22) Filed: Apr. 22, 2004

(51) Int. Cl. A61B 5/04

(52) U.S. Cl. 600/520

(57) ABSTRACT

The invention provides a method and system for bringing an exercising human subject to the state of optimal psycho-physical functioning, in effect the “runner's high”, by facilitating the state of heart rate variability coherence while the subject is exercising. This is achieved by synchronization of the varying breathing cycle with the varying heart rate variability cycle. This is accomplished by providing a biofeedback signal to the exercising subject indicating when to begin inhalation and when to begin exhalation. Additionally, the human subject is provided with a programmable tempo that is in keeping with the heart rate variability cycle to which their physical motion may be synchronized. An instructive method in the optimal application of the present invention is also provided.

### RELATED PATENTS

Method and System for Consciously Synchronizing the Breathing Cycle with the Natural Heartbeat rate Cycle (Ser. No. 10/699,025), Method and System of Respiratory Therapy Employing Heart Rate Variability Coherence (March 2004), Method and System Providing A Fundamental Musical Interval for Heart Rate Variability Synchronization (March 2004)

### FIELD OF THE INVENTION

The present invention relates to human physiology, in particular, to a method and system for allowing a human subject to consciously control physiological processes. More particularly, it allows a human subject to achieve the state of heart rate variability coherence during exercise.

### BACKGROUND OF THE INVENTION

The human heart is known to have its own nervous system and its own natural tendency toward rhythm. For purposes of this invention, there are two primary aspects to this rhythm, the heartbeat rate, and the rate at which the heartbeat rate changes otherwise known as heart rate variability. Heartbeat rate is usually specified in absolute number of heartbeats occurring during a specified period. Heart rate variability, otherwise know as heart rate variability is the change in heartbeat rate as occurs during a specified period. Henceforth, heartbeat rate variability will be referred to as heart rate variability.

It is generally recognized that heart rate variability is an indicator of physiological and emotional state, that is, irregular incoherent heart rate variability indicates a condition of physiological/psychological stress and coherent heart rate variability indicates a condition of physiological psychological harmony. Both the heartbeat rate and the period of the heart rate variability cycle vary with physical activity such that as physical activity increases, heartbeat rate and the frequency of the heart rate variability cycle increase.

The present patent asserts that the highly elusive “runner's high” is in actuality the state of heart rate variability coherence, this state being achieved during exercise as opposed to rest. Because coherence of the heart rate variability cycle results in an optimal psycho-physiological state, including cardio-vascular efficiency, the exerciser is able to exercise longer, faster, harder, and more comfortably than otherwise. Per prior invention, Method and System for Consciously Synchronizing the Breathing Cycle with the Natural Heartbeat rate Cycle (Ser. No. 10/699,025), coherence of the heart rate variability cycle may be achieved by consciously synchronizing the breathing cycle with the heart rate variability cycle.

### SUMMARY OF THE INVENTION

A relationship exists between the heartbeat rate specified in terms of heart rate variability, and the breathing cycle. While the heart has its own tendency toward a natural variable rhythm, there is a strong yet plesiochronous relationship with breathing according to this specific relationship: as inhalation occurs, there is a tendency for the heartbeat rate to increase, as exhalation occurs, there is a tendency for the heartbeat rate to decrease. For the state of heart rate variability coherence to be achieved, it is necessary for the heart rate variability cycle and the breathing cycle to be synchronized.

As the rate of exercise is increased, several physiological changes occur, the most notable of which are an increase in heartbeat rate, an increase in the frequency of breathing, and an increase in the frequency of the heart rate variability cycle, i.e. a shortening of the period of the heart rate variability cycle. As previously mentioned, the relationship between breathing and heart rate variability is a plesiochronous one, that is, while the heart rate variability cycle and the breathing cycle highly influence each other toward synchrony, in general, moments of synchronization are incidental.

When an exerciser settles into a stable pace within their ability, both their heartbeat and their natural heart rate variability rates stabilize. But, because their natural heart rate variability cycle and their breathing cycles align only incidentally, a significant degree of heart rate variability coherence is not achieved and the exerciser often does not attain the elusive “runner's high” psycho-physiological state. Alternatively, with the application of the present invention, when the heart rate variability cycle is stable, it is possible to consciously synchronize the breathing cycle with the heart rate variability cycle, thereby achieving the state of heart rate variability coherence.

Prior to the present invention, an exerciser experiences “runner's high” only when they are able to completely let go of all conscious control of breathing and bodily synchronization in which case both breathing and movement align with the heart rate variability cycle. A common mistake that many exercisers make is that they consciously synchronize their breathing with their bodily movement or visa versa. This results in a two separate rhythms that align only incidentally. Alternatively, the present invention, instructs the exerciser to first align their breathing cycle with their heart rate variability cycle and then to align their movement with their breathing cycle. This results in an overall synchronization of heart rate variability, breathing, and motion yielding the optimal psycho-physiological performance.

In summary, the present invention allows an exerciser to reliably achieve the elusive “runner's high” by consciously synchronizing their breathing cycle with their heart rate variability cycle while exercising. To accomplish this, the exerciser's heart rate variability cycle is monitored during exercise. Positive and negative heartbeat rate peaks are identified such that the exerciser may synchronize their inhalation with the positive going phase and their exhalation with the negative going phase of the heart rate variability cycle. This basic function is explained in detail in Method and System for Consciously Synchronizing the Breathing Cycle with the Natural Heartbeat rate Cycle (Ser. No. 10/699,025). Upon consciously synchronizing their breathing cycle with their heart rate variability cycle in this manner, coherence of their heart rate variability cycle is achieved and the optimal psycho-physiological “runner's high” results.

### BRIEF DESCRIPTION OF THE DRAWING FIGURES

The accompanying drawing figures incorporated in and forming a part of this specification illustrate several aspects of the invention, and together with the description serve to explain the principles of the invention.
FIG. 1 depicts the relative relationship between the heart rate variability cycle and breathing cycle.
FIG. 2 depicts the natural heart rate variability cycle and breathing cycle moving from misalignment to alignment and resultant heart rate variability patter.
FIG. 3 depicts the system block diagram of the preferred embodiment of the present invention.

FIG. 4 provides a more detailed system block diagram and description of the tempo generation function.

### DETAILED DESCRIPTION OF THE PREFERRED EMBODIMENTS

The embodiments set forth below represent the necessary information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

The present invention allows a human subject to achieve coherence of heart rate variability while exercising by synchronizing their varying breathing cycle with their varying heart rate variability cycle. This is accomplished by outfitting the exerciser with a heartbeat rate monitor that discriminates both average heartbeat rate stability and peak positive and negative heartbeat rates. The exerciser is instructed to begin inhalation coincident with peak negative heartbeat rate and begin exhalation coincident with peak positive heartbeat rate.

With reference to FIG. 1, the heart has its own nervous system and a tendency toward its own natural rhythm. FIG. 1 depicts both the heart rate variability cycle 101 and the breathing cycle 102. For the purposes of discussion, FIG. 1 defines the peak positive heartbeat rate as 80 beats per minute (BPM) 103 and the peak negative rhythm as 50 beats per minute (BPM) 104. Let it be dear that 80 beats per minute as the positive peak and 50 beats per minute as the negative peak are merely used for purposes of example and that the heartbeat rate is assumed to be completely variable depending on the state of the exerciser moment by moment. The breathing cycle is under control of the human central nervous system and interacts with the heart rate variability cycle in a plesiochronous fashion. As previously described there is a strong correlation between the breath cycle 102 and the natural heart rate variability cycle 101 as will be discussed in more detail in FIG. 2.

With reference now to FIG. 2, synchrony between the natural heart rate variability cycle 202, and the breathing cycle 201, is highly variable ranging from being highly synchronous (in-phase) to being highly asynchronous (out of phase). This results in a highly periodic and coherent heart rate variability pattern 205 vs. a highly aperiodic and incoherent heart rate variability pattern 204, respectively. The primary application of the present invention is to lead a human subject to the preferred state of highly periodic and coherent heart rate variability, 205 during exercise. This coherence is accomplished by consciously synchronizing the breathing cycle with the heart rate variability cycle.

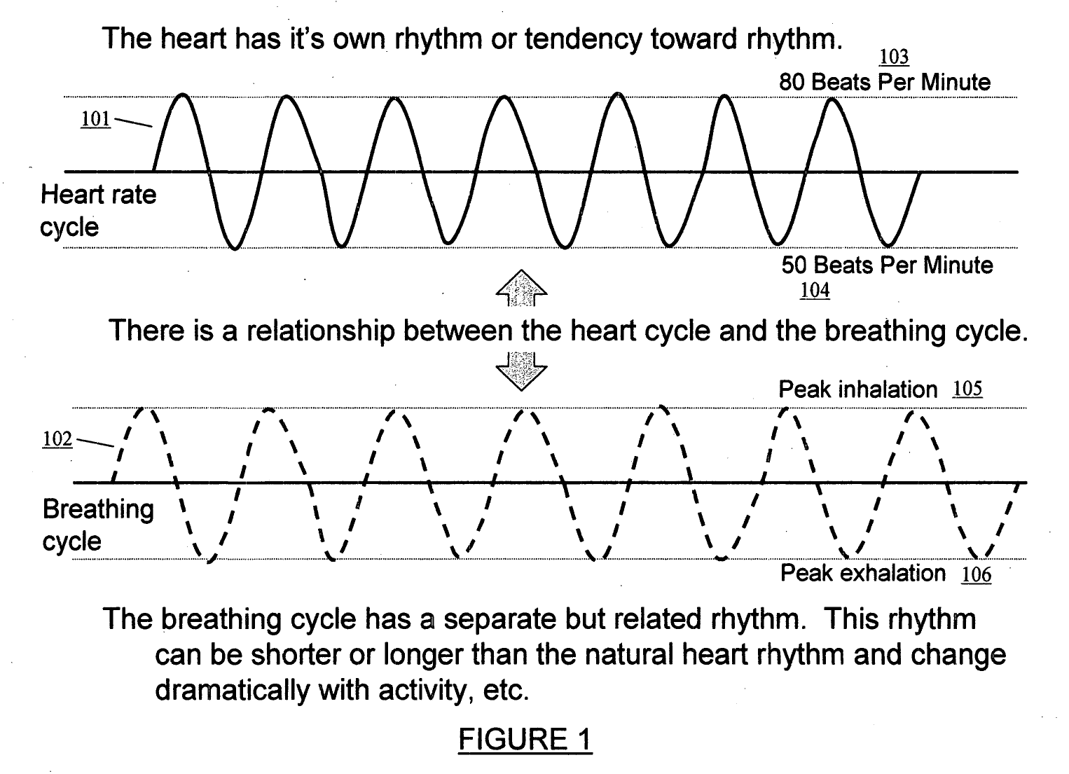

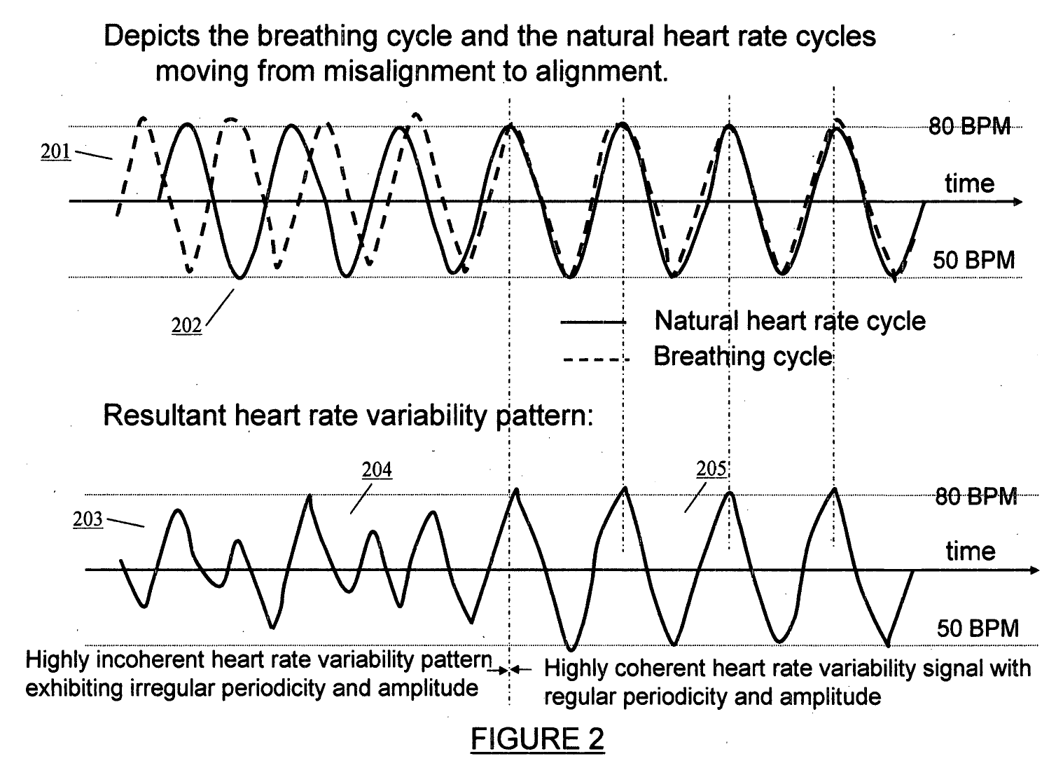

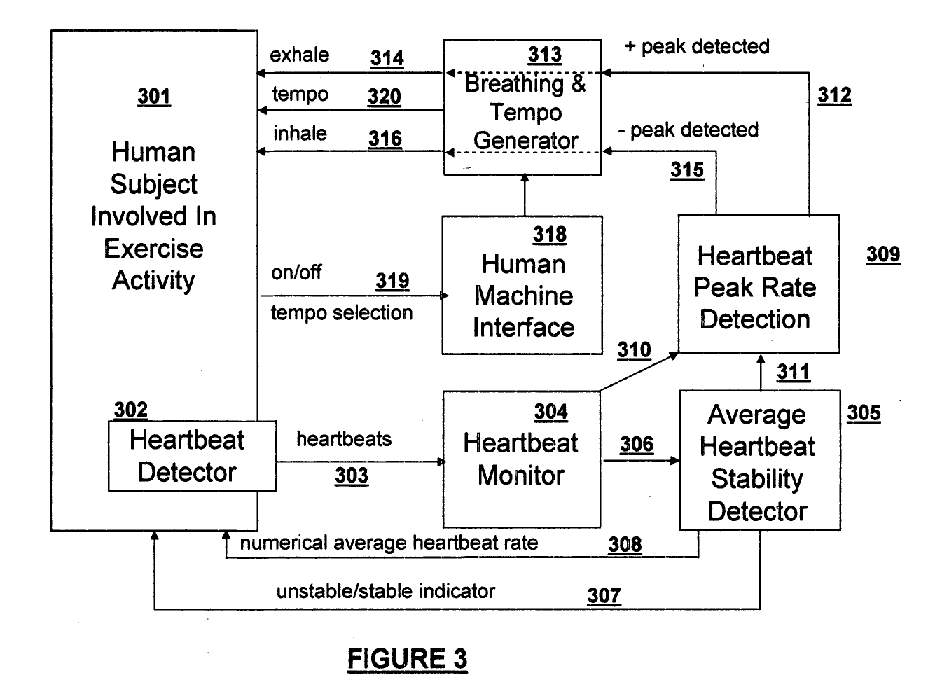

FIG. 3 depicts the logical system wherein the present invention is embodied. With reference now to FIG. 3, human subject 301 is outfitted with heartbeat detector 302. Heartbeat detector 302, detects and transduces heartbeats from their electrical or mechanical form into a form suitable for presentation to heartbeat monitor 304. Heartbeat detector 302 is assumed to be of any physical form and may be physically separate or integrated with the rest of the system. Heartbeat monitor 304 buffers and accurately reproduces heartbeats arriving from heartbeat detector 302 via connector 303. Average heartbeat stability detector 305 monitors the output of heartbeat monitor 304 via connector 306 to detect when the average heartbeat stabilizes. This stability is indicative that the exerciser has reached a certain plateau relative to their exercise rate and that their heart and breathing rate have adjusted to this plateau. This stability or lack thereof is communicated to human subject 301 via connector 307 for the purpose of indicating that breathing cycle may or may not be synchronized. A second output is provided to human subject 301 via connector 308 which numerically presents the average heartbeat rate. A second output of heartbeat monitor 304 connects to heartbeat peak rate detector 309 via connector 310. When enabled by heartbeat stability detector 305 via connector 311, heartbeat peak rate detector 309 monitors and processes the output of heartbeat monitor 304 via connector 310 for positive and negative heartbeat rate peaks and interval. When the peak positive heartbeat rate is detected, heartbeat peak rate detector 309 outputs a signal via connector 312 to breathing and tempo generator 313 for purposes of generating a unique signal via connector 314 to human exerciser 301 to begin exhalation. Alternatively, when the peak negative heartbeat rate is detected, heartbeat peak rate detector 309 outputs a signal via connector 315 to breathing and tempo generator 313 for purposes of generating a second unique signal via connector 316 to human exerciser 301 to begin inhalation. It is assumed that this feedback may take any form including audible, visual, and tactile forms or any combination of the three. This process continues for as long as the average heartbeat rate remains stable.

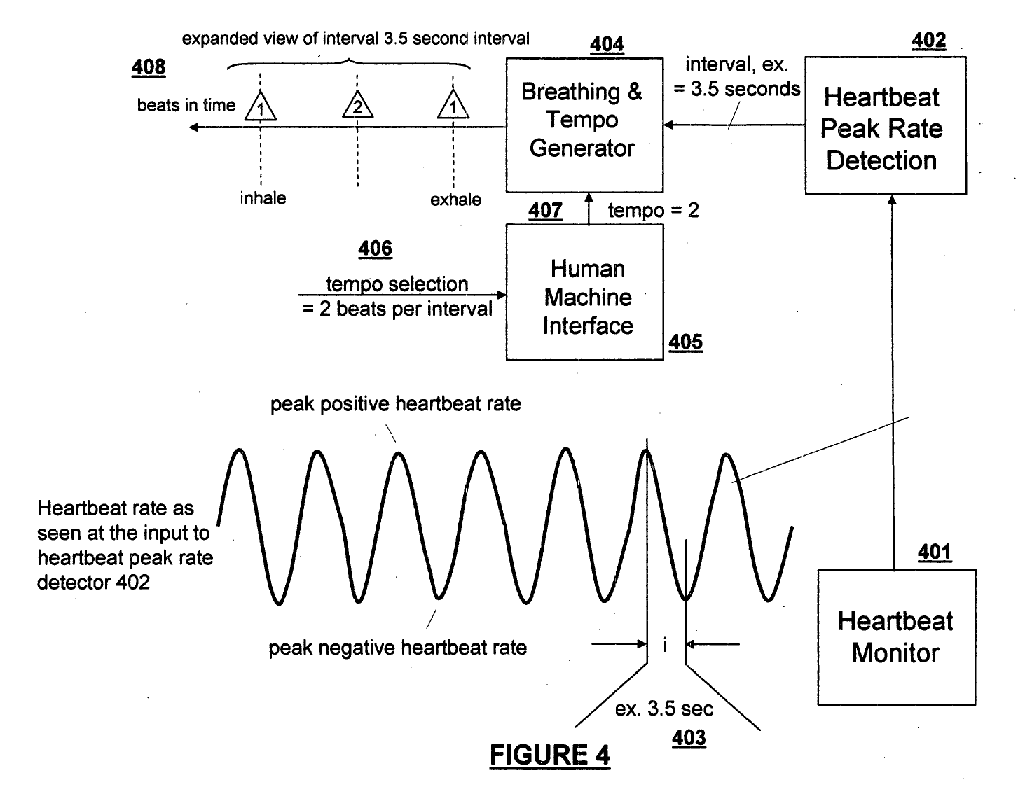

Optionally, the capability is provided to generate a completely varying tempo based on the interval between inhalation and exhalation as is computed by heartbeat peak rate detector 309 and presented to breathing and tempo generator 313 via connector 321. This tempo may vary from 1 to N beats within a practical range such that the exerciser may pace their movement according to the tempo, the tempo being in keeping with both the breathing and the heart rate variability cycle. Examples of how the tempo may be used are “4 strokes per inhalation or exhalation” or “2 rows per inhalation or exhalation”, etc. This tempo is programmable via human machine interface 318, is generated by breathing and tempo generator 313 based on the interval computed by heartbeat peak rate detector 309, and is delivered to human subject 301 via connector 320. Typically, the desired tempo is programmed at the beginning of exercise. Tempo generation is explained in more detail relative to FIG. 4 and related discussion.

If exerciser 301 decides to increase or decrease their pace, the average heartbeat rate will increase or decrease respectively during which time, human subject 301 will be alerted via connector 307 that their heartbeat rate is unstable and consequently, that feedback as to when to inhale and exhale is disabled. During these transitional periods the exerciser is also able to observe that their average heartbeat rate is changing via connector 308. Turning feedback on and off as well as setting other feedback parameters is accomplished via human machine interface 318 via connector 319.

Referring now to FIG. 4 and the function of tempo generation, as previously discussed, the output of heartbeat monitor 401 is presented to heartbeat peak rate detector 402 for purposes of discriminating peak heartbeat rates and the interval between positive and negative heartbeat rate peaks. For purposes of example, the interval between positive and negative heartbeat rate peaks is assumed to be 3.5 seconds 403. This interval is discerned by heartbeat peak rate detector 402 and transmitted to breathing and tempo generator 404 for purposes of generating a tempo under control of human machine interface 405, in this case 2 beats per interval 406. This tempo selection is transmitted to breathing and tempo generator via connector 407. Breathing and tempo generator 404 divides the interval by the number of beats and generates a corresponding signal 408 to the human subject. This signal is assumed to be in audible, visual, or tactile form or any combination of the three.

It is assumed that the logical system may be instantiated in hardware, software, or any combination of hardware and software. Secondly, it is assumed that the invention may be instantiated any number of forms and physical packages. Thirdly, it is assumed that the invention may be incorporated into any number of existing products and form factors including fountain pens, wristwatches, exercise monitors, pedometers, speedometers, cell phones, palm top computers, personal computers, exercise machines, television sets, etc. Those skilled in the art will understand the concepts of the invention and will recognize applications of these concepts not specifically addressed herein. It should be understood that these concepts and applications fall within the scope of the disclosure and the accompanying claims.

Instructive Method:

Within the context of the present invention, an accompanying 17 step instructive method is provided

Step 1: The human subject is instructed to apply system as is appropriate to their specific exercise modality and situation. In some cases the system may be discrete as in the form of a wrist watch or it may be integrated within an exercise machine such as an exercise bicycle.

Step 2: The subject is instructed to turn on or otherwise enable the system.

Step 3: The subject is instructed to program the tempo if desired.

Step 4: If feedback is to be provided via a discrete physical component such as headphones, the human subject is instructed to apply the physical component as is appropriate.

Step 5: Prior to beginning exercise, the subject is instructed to relax and attempt to achieve a stable average heartbeat rate at which time, both “stable” and “breathing” indicators will be enabled.

Step 6: The subject is instructed to synchronize their breathing with the “breathing” indicators for a few minutes so as to become familiar with the process.

Step 7: The subject is instructed to begin exercise.

Step 8: The subject is instructed to pay attention to the “stable/unstable” indicator which is indicative that the average heartbeat rate is at a plateau and that the body has adjusted to the present pace of activity.

Step 9: The subject is instructed to observe their average heartbeat rate if so desired.

Step 10: The subject is instructed that when their average heartbeat rate becomes stable as is indicated by the “stable/unstable” indicator, they may begin to synchronize their inhalation and exhalation with the respective “breathing” indicators. As previously noted, when the average heartbeat rate is unstable “breathing” indicators are disabled.

Step 11: The subject is instructed to continue to synchronize their inhalation and exhalation with the respective “breathing” indicators for as long as they continue at the present pace of exercise.

Step 12: The subject is instructed to synchronize their body movements with the tempo for as long as their average heartbeat rate remains stable. Again an example of this may be “2 strokes per inhalation or exhalation” or “2 rows per inhalation or exhalation”.

Step 13: The subject is instructed that when they either increase or decrease their pace of exercise, that during the transitional period when their average heartbeat rate is either increasing or decreasing, that the “stable/unstable” indicator will indicate “unstable” and that “breathing” indicators will be disabled.

Step 14: At the end of the exercise period, the subject is instructed once again to relax and attempt to achieve a stable average heartbeat rate at which time both the “stable” and “breathing” indicators will be enabled.

Step 15: The subject is instructed to synchronize their breathing with the “breathing” indicators for a few minutes so as to end their exercise session in a state of heart rate variability coherence.

Step 16: The subject is instructed that via this training method, they are perfecting their ability to realize the state of heart rate variability coherence both while exercising and at while at rest.

Step 17: Finally, the subject is instructed that once they become familiar with the process via application of the present invention, they are encouraged to exercise on their own and attempt to achieve the same “runner's high” state of psycho-physiological functioning.

1. The broad method of achieving coherence of the heart rate variability cycle during exercise by facilitating the synchronization of the varying breathing cycle with the varying heart rate variability cycle for purposes of achieving the optimal psycho-physiological state and consequent optimal physical performance and experience.

2. The broad system for achieving coherence of the heart rate variability cycle during exercise by facilitating the synchronization of the varying breathing cycle with the varying heart rate variability cycle for purposes of achieving the optimal psycho-physiological state and consequent optimal physical performance and experience.

3. The method of claim 1 wherein feedback to a human subject during exercise as to when to begin inhalation and when to begin exhalation on the basis of peak negative heart rate and peak positive heart rate, respectively, is provided.

4. The system of claim 2 wherein feedback to a human subject during exercise as to when to begin inhalation and when to begin exhalation on the basis of peak negative heart rate and -peak positive heart rate, respectively, is provided.

5. The method of claim 1 wherein varying feedback as to when to inhale and when to exhale on the basis of varying heart rate variability periodicity is provided.

6. The system of claim 2 wherein varying feedback as to when to inhale and when to exhale on the basis of varying heart rate variability periodicity is provided.

7. The method of claim 1 wherein enabling and disabling feedback as to when to inhale and when to exhale on the basis of average heartbeat rate stability is provided.

8. The system of claim 2 wherein enabling and disabling feedback as to when to inhale and when to exhale on the basis of average heartbeat rate stability is provided.

9. The method of claim 1 wherein a fully variable tempo which is in keeping with the heart rate variability cycle and consequently in keeping with the breathing for purposes of synchronizing body movement with heart rate and with breathing cycles is provided.

10. The system of claim 2 wherein a fully variable tempo which is in keeping with the heart rate variability cycle and consequently in keeping with the breathing for purposes of synchronizing body movement with heart rate and with breathing cycles is provided.

11. The method of claim 1 wherein programmability of said tempo generation such that the human subject can optimize the rate of physical motion relative to their heart rate variability and breathing cycles is provided.

12. The system of claim 2 wherein programmability of said tempo generation such that the human subject can optimize the rate of physical motion relative to their heart rate variability and breathing cycles is provided.

13. The method of claim 1 wherein user selection of what feedback is desired and selection of the form of said feedback including audible, visual, and tactile forms or any combination of the three is provided.

14. The system of claim 2 wherein user selection of what feedback is desired and selection of the form of said feedback including audible, visual, and tactile forms or any combination of the three is provided.

15. The system of claim 2 wherein instantiation in hardware, software, or any combination of hardware or software is provided.

16. The system of claim 2 wherein instantiation in any and all physical form factors expressly including wristwatches, cell phones, portable sports performance devices, odometers, speedometers, exercise machines, bicycles, palm top computers, lap top computers, personal computers, television sets, etc. is provided.

17. The method of claim 1 wherein the present invention is applied to any and all exercise modalities including sport and therapeutic purposes.

18. The instructive method by which a human subject is to apply the present invention for purpose of achieving coherence of heart rate variability while exercising for the purpose of achieving the optimal psycho-physiological performance and consequent experience.
